(19) United States
(12) Patent Application Publication
(10) Pub. No.: US 2025/0186724 A1
(43) Pub. Date: Jun. 12, 2025

(54) METHOD AND SYSTEM FOR CONTROLLING TURBINE MOTOR OF VENTILATOR

(71) Applicant: RESVENT MEDICAL TECHNOLOGY CO., LTD, Shenzhen (CN)

(72) Inventors: Kuan LIU, Shenzhen (CN); Rongrong ZHU, Shenzhen (CN)

(21) Appl. No.: 18/900,676

(22) Filed: Sep. 28, 2024

(30) Foreign Application Priority Data
Dec. 11, 2023 (CN) 202311693206.2

(51) Int. Cl. A61M 16/00; G16H 40/63

(52) U.S. Cl. CPC A61M 16/026; A61M 16/0066; G16H 40/63; A61M 2016/0027; A61M 2016/0033; A61M 2205/3365

(57) ABSTRACT

A method for controlling a turbine motor of a ventilator, includes: obtaining a target rotational speed and an actual rotational speed of the turbine motor; inputting the target rotational speed and the actual rotational speed into a rotational speed active disturbance rejection controller to enable the rotational speed active disturbance rejection controller to observe and compensate disturbance to obtain a quadrature-axial current; inputting the quadrature-axial current into a current active disturbance rejection controller to enable the current active disturbance rejection controller to perform a decoupling control in a direct-axial current and the quadrature-axial current to obtain a voltage vector; and adjusting the rotational speed of the turbine motor according to the voltage vector.

### CROSS-REFERENCE TO RELATED APPLICATION

This application claims priority to Chinese Patent Application No. 202311693206.2, titled “METHOD AND SYSTEM FOR CONTROLLING TURBINE MOTOR OF VENTILATOR” and filed to the China National Intellectual Property Administration on Dec. 11, 2023, the entire contents of which are incorporated herein by reference.

### TECHNICAL FIELD

The present disclosure relates to the field of ventilators, and in particular to a method and a system for controlling a turbine motor of a ventilator.

### BACKGROUND

In modern clinical medicine, a ventilator, as an effective means that replaces autonomous ventilation, has been commonly used in treating patients who suffer from obstructive sleep apnea and chronic obstructive pulmonary disease (COPD). The ventilator occupies a very important position in the field of modern medicine.

When the ventilator is operating normally, a turbine motor is configured to provide air to the ventilator. When controlling the turbine motor, a dual proportional-integral-derivative (PID) closed-loop method may be performed to adjust a rotational speed of the turbine motor. However, since data acquisition performed by a sensor may be delayed, gas compression may occur in a ventilation pipe, and a load change may be caused at breathing of the user end. In this case, the rotational speed of the turbine motor may have a significant fluctuation and disturbance, resulting in oscillation and vibration in the entire control loop and abnormal fluctuation of the rotational speed of the turbine motor, such that the therapeutic effect is affected, and the usage experience is poor.

SUMMARY OF THE DISCLOSURE

To solve the above technical problem, the present disclosure provides a method and a system for controlling a turbine motor of a ventilator.

In a first aspect, the present disclosure provides a method for controlling a turbine motor of a ventilator, the method includes:
obtaining a target rotational speed and an actual rotational speed of the turbine motor;
inputting the target rotational speed and the actual rotational speed into a rotational speed active disturbance rejection controller to enable the rotational speed active disturbance rejection controller to observe and compensate disturbance to obtain a quadrature-axial current;
inputting the quadrature-axial current into a current active disturbance rejection controller to enable the current active disturbance rejection controller to perform a decoupling control in a direct-axial current and the quadrature-axial current to obtain a voltage vector; and
adjusting the rotational speed of the turbine motor according to the voltage vector.

In some embodiments, the operation of inputting the target rotational speed and the actual rotational speed into the rotational speed active disturbance rejection controller to enable the rotational speed active disturbance rejection controller to observe and compensate disturbance to obtain a quadrature-axial current, includes:
establishing a mathematical equation based on a basic structure and an operating principle of the turbine motor;
establishing a mathematical model of a second-order expanding state observer based on the mathematical equation;
obtaining an equation of a linear expanding state observer based on the mathematical model of the second-order expanding state observer; and
obtaining an observed rotational speed and a disturbance feedforward from the linear expanding state observer, and calculating the quadrature-axial current based on the observed rotational speed and the disturbance feedforward.

In some embodiments, the mathematical equation established based on the basic structure and the operating principle of the turbine motor is as follows:

$$J \frac{{dw}_{r}}{dt} = {T}_{e} - {T}_{L} - {Bw}_{r}$$

The J denotes a system rotational inertia; the wᵣ denotes a mechanical angle of a rotor, the Tₑ denotes an electromagnetic torque; the T_{L} denotes a load torque, and the B denotes a damping coefficient.

In some embodiments, the operation of establishing the mathematical model of the second-order expanding state observer based on the mathematical equation, includes:
establishing the rotational speed active disturbance rejection controller, obtaining a rotational speed equation based on the mathematical equation;
establishing the expanding state observer, and obtaining a standard equation based on the rotational speed equation; and
establishing the mathematical model of the second-order expanding state observer based on the standard equation.

In some embodiments, the rotational speed equation that is obtained according to the mathematical equation is as follows:

$${\dot{w}}_{r} = \frac{{K}_{c}}{J} {i}_{q}^{*} + \frac{{T}_{d}}{J} = bu + a ( x )$$

The \dot{w}ᵣ denotes a first-order derivative of wᵣ; T_{d}=K_{C}(i_{q}−i*_{q})−T_{L}−Bwᵣ; the u and the i*_{q} denote output signals of the rotational speed active disturbance rejection controller; u=i*_{q}; the b denotes a control gain, and b=K_{C}/J; the a(x) denotes an unknown part of the mathematical model, wherein a(x)=T_{d}/J.

The standard equation that is obtained based on the rotational speed equation is as follows:

$$\{ \begin{matrix}{\dot{w}}_{r} = f + {b}_{0} u \\ y = {w}_{r}\end{matrix}$$

The f=a(x)+(b−b₀)u, the f denotes a total rotational speed disturbance, the bo denotes a design parameter, the y denotes an output of the expanding state observer.

The mathematical model of the second-order expanding state observer that is established based on the standard equation is as follows:

$$\{ \begin{matrix}e = {z}_{1} - y \\ {\dot{z}}_{1} = {z}_{2} - {β}_{1} ( {z}_{1} - y ) + {b}_{0} u \\ {\dot{z}}_{2} = - {β}_{2} ( {z}_{1} - y )\end{matrix}$$

The y denotes an output of the second-order expanding state observer; the z₁ denotes an observed value [symbol] of the output y; the ż₁ denotes a first-order derivative of z₁; the z₂ denotes an observed value \hat{f} of the disturbance f; the ż₂ denotes the first-order derivative of the z₂; the e denotes an observation error; the β₁ and the β₂ denote gain parameters of the second-order expanding state observer.

In some embodiments, the method further includes:
adjusting the gain parameters β₁ and β₂ of the second-order expanding state observer.

In some embodiments, the quadrature-axial current is is obtained by performing the following equation:

$${i}_{q}^{*} = \frac{{u}_{0} - {z}_{2}}{{b}_{0}} = \frac{{k}_{p} ( {w}_{Γ}^{*} - ) - {z}_{2}}{{b}_{0}}$$

The w*ᵣ denotes the target rotational speed; the [symbol] denotes an estimated value of the rotational speed; the kₚ denotes a proportional control parameter; the z₂ denotes an observed value of the disturbance f; the b₀ denotes a to-be-configured parameter.

In some embodiments, the operation of obtaining the observed rotational speed and the disturbance feedforward from the linear expanding state observer and calculating the quadrature-axial current based on the observed rotational speed and the disturbance feedforward, includes:
obtaining the observed rotational speed and the disturbance feedforward based on the linear expanding state observer;
obtaining a following error based on a difference between the target rotational speed and the observed rotational speed; and
performing proportional control on the following error and adding the disturbance feedforward to the post-controlled following error to obtain the quadrature-axial current.

In some embodiments, the operation of obtaining the target rotational speed and the actual rotational speed of the turbine motor, includes:
obtaining flow data measured by a flow sensor and pressure data measured by a pressure sensor; and
calculating, by a microprocessor, the flow data and the pressure data to obtain the target rotational speed.

In a second aspect, the present disclosure provides a method for controlling a turbine motor of a ventilator, the method includes: a control system for a turbine motor of a ventilator, including the method of controlling the turbine motor of the ventilator as described in the first aspect.

According to the present disclosure, the rotational speed active disturbance rejection controller and the current active disturbance rejection controller are configured to control the rotational speed of the turbine motor. In this way, the rotational speed of the turbine motor of the ventilator is controlled quickly and smoothly, an impact in the control, caused by the disturbance generated when the ventilator is operating, is reduced, and precision of controlling the pressure at the user end and a response speed are improved.

### BRIEF DESCRIPTION OF THE DRAWINGS

In order to more clearly illustrate the technical solutions of the embodiments of the present disclosure, the accompanying drawings for describing the embodiments will be briefly introduced in the following. It is understood that the following accompanying drawings only show some embodiments of the present application, and therefore, shall not be regarded as a limitation of the scope of the present disclosure. Any ordinary skilled person in the art may obtain other accompanying drawings based on these drawings without making any creative work.

FIG. 1 is a flow chart of a method for controlling a turbine motor of a ventilator according to some embodiments of the present disclosure.

FIG. 2 is a schematic diagram of the method for controlling the turbine motor of the ventilator according to some embodiments of the present disclosure.

FIG. 3 is a flow chart of an operation S200 of the method according to some embodiments of the present disclosure.

FIG. 4 is a flow chart of an operation S220 of the method according to some embodiments of the present disclosure.

FIG. 5 is a structural schematic diagram of a rotational speed active disturbance rejection controller according to some embodiments of the present disclosure.

FIG. 6 is a flow chart of an operation S240 of the method according to some embodiments of the present disclosure.

FIG. 7 is a flow chart of an operation S100 of the method according to some embodiments of the present disclosure.

FIG. 8 is a structural schematic diagram of the ventilator according to some embodiments of the present disclosure.

Reference numerals in the drawings: 100—rotational speed active disturbance rejection controller; 200—current active disturbance rejection controller; 300—turbine motor; 400—flow sensor; 500—pressure sensor; 600—microprocessor.

### DETAILED DESCRIPTION

In order to make the objectives, technical solutions and advantages of the embodiments of the present disclosure clearer, the technical solutions in the embodiments of the present disclosure will be described clearly and completely in the following by referring to the accompanying drawings in the embodiments of the present disclosure. Obviously, the described embodiments are a part of, not all of, the embodiments of the present disclosure. Components of the described and illustrated embodiments shown in the accompanying drawings herein may be arranged and designed in a variety of different configurations.

Accordingly, the following detailed description of the embodiments shown in the accompanying drawings of the present disclosure is not intended to limit the claimed scope of the present disclosure, but merely indicates selected embodiments of the present disclosure. All other embodiments, which are obtained by any ordinary skilled person in the art based on the embodiments of the present disclosure without making creative work, shall fall within the scope of the present disclosure.

To be noted that similar reference numerals and letters indicate similar items in the following accompanying drawings. Therefore, once an item is defined in one accompanying drawing, the item may not be further defined and explained in subsequent accompanying drawings.

In the description of the present disclosure, to be noted that the terms “up”, “down” and the so on, are used to indicate an orientation or a positional relationship based on the orientation or the positional relationship shown in the accompanying drawings or indicate an orientation or a positional relationship in which the product of the present disclosure is commonly placed when being in use. The terms are used only for the purpose of facilitating the description of the present disclosure and simplifying the description and shall not indicate or imply that the devices or elements referred to must have a particular orientation or must be configured and operated in a particular orientation. Therefore, the terms shall not be interpreted as a limitation of the present disclosure. Furthermore, the terms “first”, “second”, and so on, are used only to distinguish descriptions and shall not be interpreted as indicating or implying relative importance.

In addition, the terms “horizontal”, “vertical”, “overhanging”, and so on, do not mean that the components are required to be absolutely horizontal or overhanging but may be slightly inclined. For example, “horizontal” simply means that an orientation is more horizontal than “vertical”, and does not mean that a structure must be perfectly horizontal, and the structure may be slightly inclined.

In the description of the present disclosure, to be noted that the terms “configured”, “mounted”, “inter-connected”, and “connected” shall be interpreted in a broad sense, unless otherwise expressly specified and qualified. For example, “connection” may be fixed connection, detachable connection, or being connected to form a one-piece structure; mechanical connection, or electrical connection; direct connection, or indirect connection through an intermediate medium; or internal connection of two elements. Any ordinary skilled person in the art shall understand the specific meaning of the above terms in the present disclosure case by case.

To be noted that different features in the embodiments of the present disclosure may be combined with each other without conflict.

Specific embodiments of the present disclosure are described in detail below by referring to the accompanying drawings.

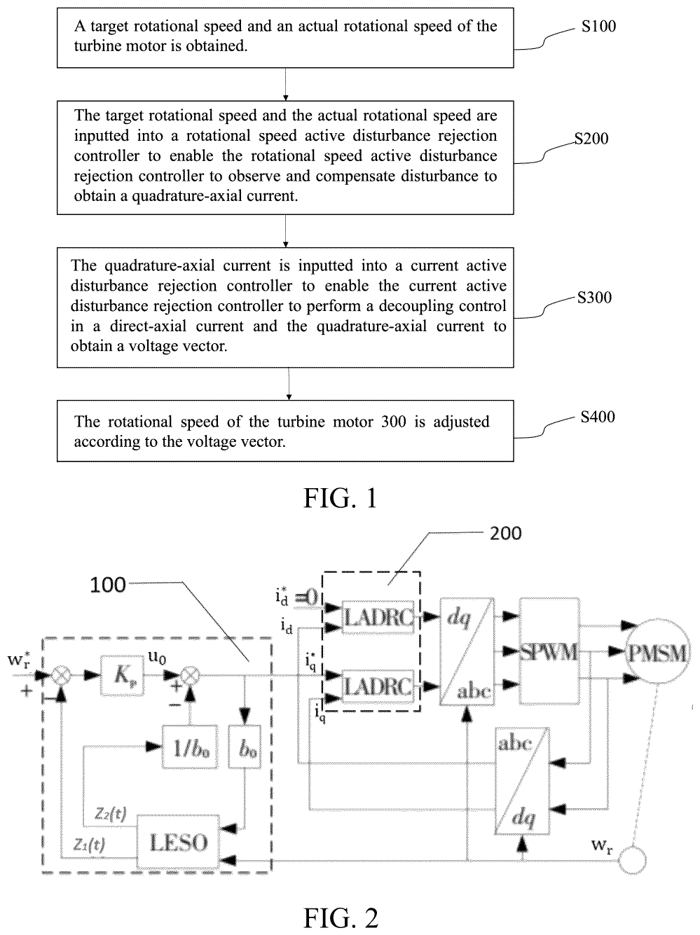

As shown in FIG. 1, the present disclosure provides a method for controlling a turbine motor of a ventilator. The method is applied to a ventilator. The method for controlling the turbine motor of the ventilator includes the following operations.

In an operation S100, a target rotational speed and an actual rotational speed of the turbine motor 300 is obtained.

In the present embodiment, the target rotational speed is obtained by calculation based on data measured by a flow sensor 400 and data measured by a pressure sensor 500. At the same time, the actual rotational speed is also obtained by the measurement performed by sensors in the turbine motor 300.

In an operation S200, the target rotational speed and the actual rotational speed are inputted into a rotational speed active disturbance rejection controller 100 to enable the rotational speed active disturbance rejection controller 100 to observe and compensate disturbance to obtain a quadrature-axial current.

In the present embodiment, the target rotational speed and the actual rotational speed obtained at the operation S100 are inputted into the rotational speed active disturbance rejection controller 100. The rotational speed active disturbance rejection controller 100 observes and compensates, in real time, the disturbance caused by delay and load variations generated during the sensors in the ventilator obtaining the data, and the quadrature (Q)-axial current is obtained.

Specifically, as shown in FIG. 2, the rotational speed active disturbance rejection controller 100 includes one second-order expanding state observer and one first-order linear state error feedback.

In an operation S300, the quadrature-axial current is inputted into a current active disturbance rejection controller 200 to enable the current active disturbance rejection controller 200 to perform a decoupling control in a direct-axial current and the quadrature-axial current to obtain a voltage vector.

In the present embodiment, a given direct (D)-axial current and the quadrature (Q)-axial current obtained at the operation S200 are inputted into the current active disturbance rejection controller 200. The current active disturbance rejection controller 200 performs the decoupling control in the direct-axial current and the quadrature-axial current, and the voltage vector is obtained.

Specifically, as shown in FIG. 2, the current active disturbance rejection controller 200 includes two linear active disturbance rejection controller (LADRC) that are identical to each other. The given direct (D) axial current may be 0, and that is, i*_{d}=0.

id\hat{ }*=0.

In an operation S400, the rotational speed of the turbine motor 300 is adjusted according to the voltage vector.

In the present embodiment, the rotational speed of the turbine motor 300 is controlled according to the voltage vector obtained at the operation S300 to minimize an impact, caused by the disturbance generated when the ventilator is operating, in the control of the rotational speed.

In the above configuration, the rotational speed of the turbine motor 300 is controlled by the rotational speed active disturbance rejection controller 100 and the current active disturbance rejection controller 200. The rotational speed of the turbine motor 300 of the ventilator is controlled quickly and smoothly. The impact, caused by the disturbance generated when the ventilator is operating, in controlling the rotational speed is reduced, the precision of controlling the pressure at the user end and the response speed are improved.

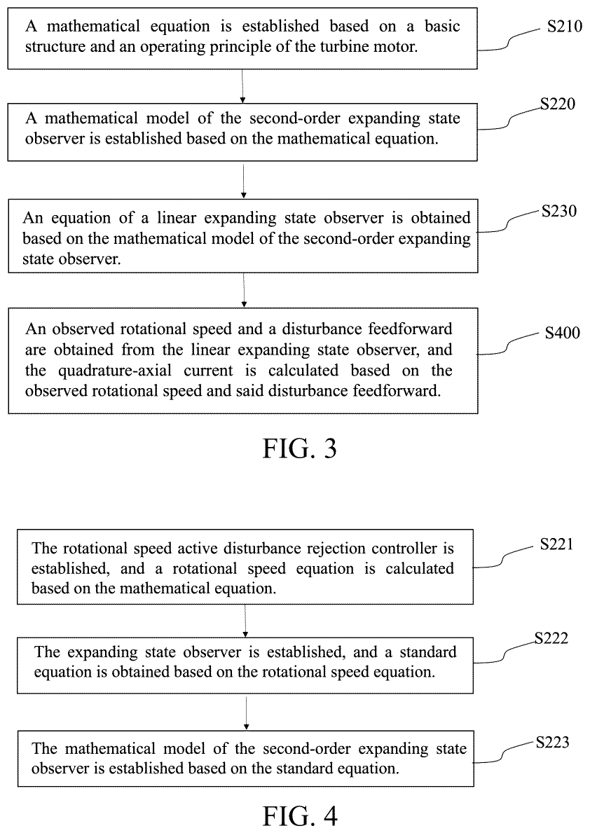

In an embodiment, as shown in FIG. 3, the operation S200 includes the following sub-operations.

In a sub-operation S210, a mathematical equation is established based on a basic structure and an operating principle of the turbine motor 300.

In the present embodiment, a mechanical motion equations of the turbine motor 300 is established based on the basic structure and the operating principle of the turbine motor 300.

Specifically, the established mathematical equation is as follows:

$$J \frac{{dw}_{r}}{dt} = {T}_{e} - {T}_{L} - {Bw}_{r}$$

The J denotes a system rotational inertia; the wr denotes a mechanical angle of a rotor, the Tₑ denotes an electromagnetic torque; the T_{L} denotes a load torque, and the B denotes a damping coefficient.

The electromagnetic torque Tₑ is obtained by performing the following equation:

Tₑ=1.5pₙ [symbol] _{f}i_{q}

The pₙ denotes the number of pole pairs of the motor; the [symbol] _{f} denotes a magnetic chain of the motor; and the i_{q} denotes the Q-axial current of the motor.

In a sub-operation S220, a mathematical model of the second-order expanding state observer is established based on the mathematical equation.

In the present embodiment, the second-order expanded state observer mathematical model is constructed based on the mathematical equation established at the sub-operation S210.

In a sub-operation S230, an equation of a linear expanding state observer is obtained based on the mathematical model of the second-order expanding state observer.

In the present embodiment, the equation of the linear expanding state observer is obtained based on the mathematical model of the second-order expanding state observer established at the sub-operation S230.

In a sub-operation S240, an observed rotational speed and a disturbance feedforward are obtained from the linear expanding state observer, and the quadrature-axial current is calculated based on the observed rotational speed and said disturbance feedforward.

In the present embodiment, the quadrature-axial current i*_{q} is calculated based on the observed rotational speed [symbol] and the disturbance feedforward \hat{f} that are obtained from the linear expanding state observer.

In an implementation, as shown in FIG. 4, the sub-operation S220 includes following sub-operations.

In a sub-operation S221, the rotational speed active disturbance rejection controller 100 is established, and a rotational speed equation is calculated based on the mathematical equation.

In the present embodiment, the rotational speed equation obtained according to the mathematical equation is as follows:

$${\dot{w}}_{r} = \frac{{K}_{c}}{J} {i}_{q}^{*} + \frac{{T}_{d}}{J} = bu + a ( x )$$

The \dot{w}ᵣ denotes a first-order derivative of wᵣ; T_{d}=K_{C}(i_{q}−*_{q})−T_{L}−Bwᵣ; the u and the i*_{q} denote output signals of the rotational speed active disturbance rejection controller 100; u=i*_{q}; the b denotes a control gain; and b=K_{C}/J; the a (x) denotes an unknown part of the model (the disturbance); and a(x)=T_{d}/J.

In a sub-operation S222, the linear expanding state observer (LESO) is established, and a standard equation is obtained based on the rotational speed equation.

In the present embodiment, the standard equation obtained based on the rotational speed equation is as follows:

$$\{ \begin{matrix}{\dot{w}}_{r} = f + {b}_{0} u \\ y = {w}_{r}\end{matrix}$$

In the above equation, f=a(x)+(b−b₀) u, the f denotes a total rotational speed disturbance, the b₀ denotes a design parameter, the y denotes an output of the expanding state observer.

In a sub-operation S223, the mathematical model of the second-order expanding state observer is established based on the standard equation.

In the present embodiment, the mathematical model of the second-order expanding state observer that is established based on the standard equation is as follows:

$$\{ \begin{matrix}e = {z}_{1} - y \\ {\dot{z}}_{1} = {z}_{2} - {β}_{1} ( {z}_{1} - y ) + {b}_{0} u \\ {\dot{z}}_{2} = - {β}_{2} ( {z}_{1} - y )\end{matrix}$$

The y denotes an output of the second-order expanding state observer; the z₁ denotes an observed value [symbol] of the output y; the ż₁ denotes a first-order derivative of z₁; the z₂ denotes an observed value \hat{f} of the disturbance f; the ż₂ denotes the first-order derivative of the z₂; the e denotes an observation error; the β₁ and the β₂ denote gain parameters of the second-order expanding state observer.

The second-order expanding state observer estimates, in real time, a state and the disturbance, i.e.,:

$$\{ \begin{matrix}{z}_{1} \to \\ {z}_{2} \to \hat{f}\end{matrix}$$

The [symbol] denotes the observed value of the output y; and the \hat{f} denotes an observed value of the disturbance f.

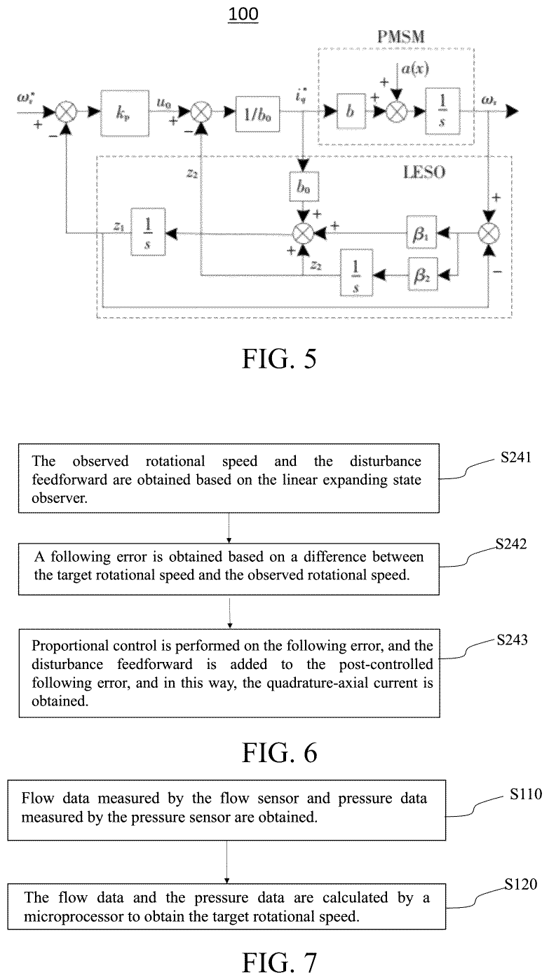

In a sub-operation S230, as shown in FIG. 5, a new output signal uₒ is set, in which

$$u = \frac{{u}_{o} - {z}_{2}}{{b}_{o}} ,$$

and the system is equivalent to a linear system, and that is:

\dot{w}ᵣ=u₀

The disturbance can be canceled out by the expanding state observer. Therefore, a better control effect can be achieved by only a proportional control (P).

In an implementation, in the operation S200, the gain parameters β₁ and β₂ of the second-order expanding state observer can be adjusted.

In the present embodiment, the gain parameters β₁ and β₂ of the second-order expanding state observer can be configured via a broadband of the observer wₒ, as follows:

$$\{ \begin{matrix}{β}_{1} = 2 {w}_{o} \\ {β}_{2} = {w}_{o}^{2}\end{matrix}$$

The wₒ denotes the broadband of the observer and can be adjusted according to the actual situation.

Since \dot{w}ᵣ=u₀, a closed-loop transfer function of the system is as follows:

$${G}_{e} ( s ) = \frac{{k}_{p}}{s + {k}_{p}}$$

The kₚ denotes a proportional control parameter; s=σ+jw, and the s is a complex variable; the real part σ∈R+; the imaginary part w∈R; and the R and the R+ denote a real domain and a positive real domain, respectively.

The proportional control parameter kₚ can be configured through the broadband of the controller w_{c}, as follows:

kₚ=w_{c}

The w_{c} denotes the broadband of the controller, which can be adjusted according to the actual situation.

In the above configuration, the broadband of the observer wₒ and the broadband of the controller w_{c} need to be adjusted. The broadband of the observer wₒ may be three times of the broadband of the controller w_{c}, i.e., wₒ=3w_{c}. Therefore, only the broadband of the controller w_{c} needs to be adjusted. By adjusting the broadband of the controller w_{c}, the controller is stabilized and converged. For example, for a given rotational speed, the output of the rotational speed active disturbance rejection controller 100 is the quadrature (Q)-axial current. The quadrature (Q)-axial current may be a fixed unknown value. By adjusting the parameter, the quadrature (Q)-axial current is obtained more quickly and more accurately.

In an implementation, since the disturbance is already canceled by the expanding state observer (LESO), the better control can be achieved by performing only the proportional control (P), without performing the integral control (I). The quadrature (Q)-axial current i*_{q} is obtained by performing the following equation:

$${i}_{q}^{*} = \frac{{u}_{0} - {z}_{2}}{{b}_{0}} = \frac{{k}_{p} ( {w}_{Γ}^{*} - ) - {z}_{2}}{{b}_{0}}$$

The w*ᵣ denotes the target rotational speed; the [symbol] denotes an estimated value of the rotational speed; the kₚ denotes the proportional control parameter; the z₂ denotes an observed value of the disturbance f; the b₀ denotes a to-be-configured parameter, which can be adjusted according to the actual situation.

In the above configuration, the broadband of the observer wₒ, the broadband of the controller w_{c}, and the to-be-configured parameter bo need to be adjusted. The broadband of the observer wₒ can be three times of broadband of the controller w_{c}, i.e., wₒ=3w_{c}. Therefore, only the broadband of the controller w_{c} and the to-be-configured parameter b₀ need to be adjusted.

In an implementation, as shown in FIG. 6, the operation S240 includes the following sub-operations.

In a sub-operation S241, the observed rotational speed and the disturbance feedforward are obtained based on the linear expanding state observer.

In a sub-operation S242, a following error is obtained based on a difference between the target rotational speed and the observed rotational speed.

In a sub-operation S243, proportional control is performed on the following error, and the disturbance feedforward is added to the post-controlled following error, and in this way, the quadrature-axial current is obtained.

In the present embodiment, as shown in FIG. 5, the observed rotational speed z₁ and the disturbance feedforward z₂ are obtained based on the linear expanding state observer. The following error is obtained based on the difference between the target rotational speed w*ᵣ and the observed rotational speed z₁. The proportional control is performed on the following error, the disturbance feedforward z₂ is added to the post-controlled following error, and the quadrature-axial current i*_{q} is obtained.

In an implementation, as shown in FIG. 7, the operation S100 includes the following sub-operations.

In a sub-operation S110, flow data measured by the flow sensor 400 and pressure data measured by the pressure sensor 500 are obtained.

In a sub-operation S120, the flow data and the pressure data are calculated by a microprocessor 600 to obtain the target rotational speed.

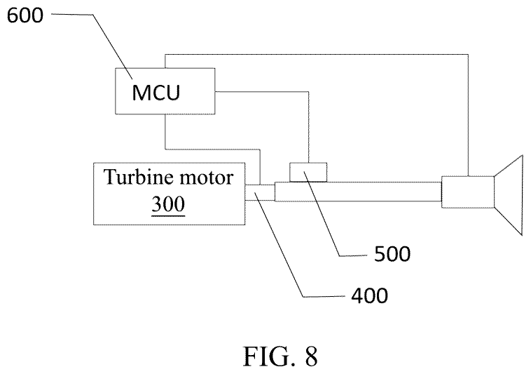

In the present embodiment, as shown in FIG. 8, the ventilator includes the turbine motor 300, the flow sensor 400, the pressure sensor 500, and the microprocessor 600 (MCU). The flow sensor 400 is configured to measure the flow data at a breathing end of the ventilator. The pressure sensor 500 is configured to measure pressure data at the breathing end of the ventilator. The microprocessor 600 (MCU) is connected to the flow sensor 400 and the pressure sensor 500. The microprocessor 600 (MCU) is configured to perform calculations on the flow data the pressure data.

Specifically, after the flow data is obtained by the flow sensor 400 and the pressure data is obtained by the pressure sensor 500, the flow data and the pressure data are calculated by the microprocessor 600 (MCU) to obtain the target rotational speed w*ᵣ.

Embodiments of the present disclosure further provide a control system of the turbine motor of the ventilator. The control system includes the method for controlling the turbine motor of the ventilator as described in the above.

In summary, the present disclosure provides a method and a system for controlling the turbine motor of the ventilator. The rotational speed of the turbine motor is controlled by the rotational speed active disturbance rejection controller and the current active disturbance rejection controller. In this way, the rotational speed of the turbine motor of the ventilator is controlled quickly and smoothly, the impact, caused by the disturbance generated when the ventilator is operating, in controlling the rotational speed is reduced, and precision of controlling the pressure at the user end and a response speed are improved.

To be noted that, in the present disclosure, relational terms, such as first and second, are used only to distinguish one entity or one operation from another, and do not necessarily require or imply existence of any actual relationship or order between the entities or operations. Furthermore, the terms “include”, “contain”, and any other variant thereof, are intended to cover non-exclusive inclusion. Therefore, a process, a method, an article, or an apparatus including a set of elements includes not only the listed elements, but further includes other elements that are not expressly listed or other elements that are inherently included in the process, the method, the article or the apparatus. Without further limitation, elements defined by the phrase “include . . . ” do not preclude existence of additional identical elements in the process, the method, the article or the apparatus.

The above description describes only preferred embodiments of the present disclosure and does not limit the present disclosure. For any ordinary skilled person in the art, the present disclosure may have changes and variations. Any modifications, equivalent substitutions, and improvements made within the concept and principles of the present disclosure shall be included in the scope of the present disclosure.

What is claimed is:

1. A method for controlling a turbine motor of a ventilator, comprising:
obtaining a target rotational speed and an actual rotational speed of the turbine motor;
inputting the target rotational speed and the actual rotational speed into a rotational speed active disturbance rejection controller to enable the rotational speed active disturbance rejection controller to observe and compensate disturbance to obtain a quadrature-axial current;
inputting the quadrature-axial current into a current active disturbance rejection controller to enable the current active disturbance rejection controller to perform a decoupling control in a direct-axial current and the quadrature-axial current to obtain a voltage vector; and
adjusting the rotational speed of the turbine motor according to the voltage vector.

2. The method according to claim 1, wherein, the operation of inputting the target rotational speed and the actual rotational speed into the rotational speed active disturbance rejection controller to enable the rotational speed active disturbance rejection controller to observe and compensate disturbance to obtain a quadrature-axial current, comprises:
establishing a mathematical equation based on a basic structure and an operating principle of the turbine motor;
establishing a mathematical model of a second-order expanding state observer based on the mathematical equation;
obtaining an equation of a linear expanding state observer based on the mathematical model of the second-order expanding state observer; and
obtaining an observed rotational speed and a disturbance feedforward from the linear expanding state observer, and calculating the quadrature-axial current based on the observed rotational speed and the disturbance feedforward.

3. The method according to claim 2, wherein, the mathematical equation established based on the basic structure and the operating principle of the turbine motor is as follows:
$$J \frac{{dw}_{r}}{dt} = {T}_{e} - {T}_{L} - {Bw}_{r}$$
wherein, the J denotes a system rotational inertia; the wᵣ denotes a mechanical angle of a rotor, the Tₑ denotes an electromagnetic torque; the T_{L} denotes a load torque, and the B denotes a damping coefficient.

4. The method according to claim 2, wherein, the operation of establishing the mathematical model of the second-order expanding state observer based on the mathematical equation, comprises:
establishing the rotational speed active disturbance rejection controller, obtaining a rotational speed equation based on the mathematical equation;
establishing the expanding state observer, and obtaining a standard equation based on the rotational speed equation; and
establishing the mathematical model of the second-order expanding state observer based on the standard equation.

5. The method according to claim 4, wherein, the rotational speed equation that is obtained according to the mathematical equation is as follows:
$${\dot{w}}_{r} = \frac{{K}_{c}}{J} {i}_{q}^{*} + \frac{{T}_{d}}{J} = bu + a ( x )$$
wherein, the \dot{w}ᵣ denotes a first-order derivative of wᵣ; T_{d}=K_{C}(i_{q}−i*_{q})−T_{L}−Bwᵣ; the u and the i*_{q} denote output signals of the rotational speed active disturbance rejection controller; u=i*_{q}; the b denotes a control gain, and b=K_{C}/J; the a(x) denotes an unknown part of the mathematical model, wherein a(x)=T_{d}/J;
the standard equation that is obtained based on the rotational speed equation is as follows:
$$\{ \begin{matrix}{\dot{w}}_{r} = f + {b}_{0} u \\ y = {w}_{r}\end{matrix}$$
wherein, f=a(x)+(b−b₀)u, the f denotes a total rotational speed disturbance, the b₀ denotes a design parameter, the y denotes an output of the expanding state observer;
the mathematical model of the second-order expanding state observer that is established based on the standard equation is as follows:
$$\{ \begin{matrix}e = {z}_{1} - y \\ {\dot{z}}_{1} = {z}_{2} - {β}_{1} ( {z}_{1} - y ) + {b}_{0} u \\ {\dot{z}}_{2} = - {β}_{2} ( {z}_{1} - y )\end{matrix}$$
wherein, the y denotes an output of the second-order expanding state observer; the z₁ denotes an observed value [symbol] of the output y; the ż₁ denotes a first-order derivative of z₁; the z₂ denotes an observed value \hat{f} of the disturbance f; the ż₂ denotes the first-order derivative of the z₂; the e denotes an observation error; the β₁ and the β₂ denote gain parameters of the second-order expanding state observer.

6. The method according to claim 5, wherein, the method further comprises:
adjusting the gain parameters β₁ and the β₂ of the second-order expanding state observer.

7. The method according to claim 1, wherein, the quadrature-axial current is is obtained by performing the following equation:
$${i}_{q}^{*} = \frac{{u}_{0} - {z}_{2}}{{b}_{0}} = \frac{{k}_{p} ( {w}_{r}^{*} - ) - {z}_{2}}{{b}_{0}}$$
wherein, the w*ᵣ denotes the target rotational speed; the [symbol] denotes an estimated value of the rotational speed; the kₚ denotes a proportional control parameter; the z₂ denotes an observed value of the disturbance f; the b₀ denotes a to-be-configured parameter.

8. The method according to claim 2, wherein, the operation of obtaining the observed rotational speed and the disturbance feedforward from the linear expanding state observer and calculating the quadrature-axial current based on the observed rotational speed and the disturbance feedforward, comprises:
obtaining the observed rotational speed and the disturbance feedforward based on the linear expanding state observer;
obtaining a following error based on a difference between the target rotational speed and the observed rotational speed; and
performing proportional control on the following error and adding the disturbance feedforward to the post-controlled following error to obtain the quadrature-axial current.

9. The method according to claim 1, wherein, the operation of obtaining the target rotational speed and the actual rotational speed of the turbine motor, comprises:
obtaining flow data measured by a flow sensor and pressure data measured by a pressure sensor; and
calculating, by a microprocessor, the flow data and the pressure data to obtain the target rotational speed.

10. A control system for a turbine motor of a ventilator, comprising the method of controlling the turbine motor of the ventilator as claimed in claim 1.
